(19) United States
(12) Patent Application Publication
(10) Pub. No.: US 2022/0023904 A1
(43) Pub. Date: Jan. 27, 2022

(54) A SPRAY VEHICLE

(71) Applicants: Bayer Aktiengesellschaft, Leverkusen (DE); Monsanto Technology LLC, St Louis, MO (US)

(72) Inventors: Andrew Charles CHAPPLE, Lagenfeld (DE); Creighton DEYOUNG, Webster Groves, MO (US); Christopher LUZECKY, St. Louis, MO (US); Alexander SHERIDAN, Saint Charles, MO (US); Jeffrey KOHNE, Kirkwood, MO (US); Anthony VAUGHN, O Fallon, MO (US)

(73) Assignees: Bayer Aktiengesellschaft, Leverkusen (DE); Monsanto Technology LLC, St. Louis, MO (US)

(21) Appl. No.: 17/296,926

(22) PCT Filed: Dec. 5, 2019
(86) PCT No.: PCT/EP2019/083836

§ 371 Date: May 25, 2021

Related U.S. Application Data
(60) Provisional application No. 62/906,791, filed on Sep. 27, 2019.

(51) Int. Cl. B05B 12/12; B05B 13/00; B05B 1/20; A01M 7/00; B64C 39/02; B64D 1/18; A01H 1/02

(52) U.S. Cl. CPC B05B 12/126; B05B 13/005; B05B 1/20; A01M 7/0032; B64C 2201/12; B64C 39/024; B64D 1/18; A01H 1/027; A01M 7/0089

(57) ABSTRACT

A spray vehicle includes at least one boom, a plurality of spray units, at least one first actuator, at least one second actuator, a plurality of sensors, and a processing unit. The spray units are configured to spray pollen or a liquid chemical. The at least one first actuator is configured to move the at least one boom. The at least one second actuator is configured to control the spray units. At least one sensor is configured to measure a speed of the vehicle relative to the ground. At least one sensor is configured to measure an air movement direction relative to the vehicle. At least one sensor is configured to measure an air movement speed relative to the vehicle. The processing unit is configured to determine an air movement direction relative to the ground and determine an air movement speed relative to the ground.

### CROSS REFERENCE TO RELATED APPLICATIONS

This application is a national stage application under 35 U.S.C. § 371 of International Application No. PCT/EP2019/083836, filed internationally on Dec. 5, 2019, which claims benefit of U.S. Provisional Application No. 62/906,791, filed Sep. 27, 2019, and the benefit of European Application No. 18211247.4, filed Dec. 10, 2018.

### FIELD OF THE INVENTION

The present disclosure relates to a spray vehicle capable of spraying liquids or powders, such as pollen or a liquid chemical.

### BACKGROUND OF THE INVENTION

Spray drift caused by wind and wind gusts is a major problem in agricultural production. The spray droplets drift onto non target surfaces, such as within sensitive zones, on bystanders, onto water bodies, and onto neighboring fields.

Further, the spraying of pollen or a liquid chemical is a component of producing plants for cultivation. For example, the spraying of pollen or a liquid chemical increases in yield for crops.

### SUMMARY OF THE INVENTION

It would be advantageous to have a way of mitigating the effects of spray drift.

In an aspect, there is provided a spray vehicle, comprising:
at least one boom;
a plurality of spray units;
at least one first actuator;
at least one second actuator;
a plurality of sensors; and
a processing unit.

The at least one boom is movably attached to the vehicle. The plurality of spray units are attached to the at least one boom. The plurality of spray units are configured to spray pollen or a liquid chemical. The at least one first actuator is configured to move the at least one boom. The at least one second actuator is configured to control the plurality of spray units. At least one sensor of the plurality of sensors is configured to measure a speed of the vehicle relative to the ground. At least one sensor of the plurality of sensors is configured to measure an air movement direction relative to the vehicle. At least one sensor of the plurality of sensors is configured to measure an air movement speed relative to the vehicle. The processing unit is configured to determine an air movement direction relative to the ground and determine an air movement speed relative to the ground. The determination comprises utilization of the speed of the vehicle, the air movement direction relative to the vehicle and the air movement speed relative to the vehicle. The processing unit is configured also to control the at least one first actuator and/or the at least one second actuator. The control comprises utilization of the determined air movement direction relative to the ground and the determined air movement speed relative to the ground.

In this manner, the effect of drift of a sprayed liquid caused by wind can be mitigated through movement of a spray boom and/or control of spray units themselves, with this mitigation taking into account the real wind speed and direction. Thus, a vehicle can spray closer to the edge of fields and/or closer to footpaths, or areas that are not to be sprayed, and/or spraying can be conducted at higher vehicle speeds and at higher wind speeds than currently achievable.

In an example, the at least one first actuator is configured to move the at least one boom in a vertical direction, and wherein the processing unit is configured to control the at least one first actuator to move the at least one boom in a vertical direction.

Thus, in this way as the wind speed and/or direction would lead to more spray drift, for example in a lateral direction perpendicular to a movement direction of the spray vehicle, the boom can be lower if spray drift is expected to increase, as a consequence of the wind speed increasing and/or the wind direction moving in a direction more perpendicular to the vehicle movement direction.

In an example, at least one sensor of the plurality of sensors is configured to provide data from which a height of the at least boom above the ground can be determined. The processing unit is configured to control the at least one first actuator to position the at least one boom at a height above the ground that depends on a magnitude of the determined air movement speed relative to the ground.

In this manner, even if the ground either side of the spray vehicle is not flat, but is higher or lower than a nominal ground level, the spray booms can be positioned at the ideal position to mitigate spray drift, taking into account wind speed.

In an example, the processing unit is configured to control the at least one first actuator to position the at least one boom at a height above the ground that depends on a magnitude of an air direction angle of the determined air movement direction relative to the ground.

In this manner, even if the ground either side of the spray vehicle is not flat, but is higher or lower than a nominal ground level, the spray booms can be positioned at the ideal position to mitigate spray drift, taking into account wind speed and direction.

In an example, the air direction angle is the angle between the determined air movement direction relative to the ground and a projection of the fore-aft axis of the vehicle onto the ground.

In an example, the height above the ground at which the at least one boom is positioned is calculated on the basis of the air movement speed relative to the ground multiplied by the cosine of the air direction angle.

In other words, the effect of cross-winds or a component of wind that is perpendicular to the forward movement direction of the vehicle of spray drift can be mitigated through appropriate control of the height of a spray boom and/or control of the spray units themselves.

In an example, the processing unit is configured to control the at least one first actuator to move the at least one boom in a downwards direction when the magnitude of the determined air movement speed relative to the ground exceeds one or more threshold values.

In this manner, spraying can continue in a set manner until a wind speed exceeds a set magnitude, and then remedial action can be undertaken for this situation when spray drift can become problematic. This saves energy, saves wear on components as the boom is only moved when necessary and also mitigates the possibility of hunting, where the system could be continuously seeking an optimum setting.

In an example, the one or more threshold values depend upon a magnitude of an air direction angle, wherein the air direction angle is the angle between the determined air movement direction relative to the ground and a projection of the fore-aft axis of the vehicle onto the ground.

In an example, the one or more threshold values are a plurality of threshold values, and a threshold value of the plurality of threshold values is calculated on the basis of a set air movement speed multiplied by the cosine of the air direction angle.

In an example, the plurality of spray units are movably attached to the at least one boom, and at least one sensor of the plurality of sensors is configured to provide data from which an angle of the plurality of spray units with respect to a vertical axis can be determined. The at least one second actuator comprises at least one first rotator actuator configured to rotate the plurality of spray units by at least one angle of rotation with respect to the vertical axis. The processing unit is configured to control one or more of the at least one first rotator actuator to rotate one or more of the plurality of spray units.

In this way, the spray units can be angled to spray slightly upwind in order to mitigate wind speed and direction that could otherwise cause spray drift.

In an example, a horizontal axis extends in a direction perpendicular to a fore-aft axis of the vehicle and extends in a direction perpendicular to the vertical axis, and at least one sensor of the plurality of sensors is configured to provide data from which an angle of the plurality of spray units with respect to the horizontal axis can be determined. The at least one second actuator comprises at least one second rotator actuator configured to rotate the plurality of spray units by at least one angle of rotation with respect to the horizontal axis. The processing unit is configured to control one or more of the at least one second rotator actuator to rotate one or more of the plurality of spray units.

In an example, the processing unit is configured to control a plurality of first rotator actuators to hold an equivalent number of spray units simultaneously at different angles of rotation with respect to the vertical axis.

In an example, control of the plurality of first rotator actuators depends upon the locations of the equivalent number of spray units on the at least one boom.

Thus, spray units on the outer edge of the boom that could be nearer an edge of the field can be angled further upwind to mitigate spray drift, whilst the spray units on the other end of the boom or on the other end of a boom on the other side of the vehicle do not need to be angled to the same extent, because there is less chance of the spray from that spray unit crossing the field boundary.

In an example, the processing unit is configured to control a plurality of second rotator actuators to hold an equivalent number of spray units simultaneously at different angles of rotation with respect to the horizontal axis.

In an example, control of the plurality of second rotator actuators depends upon the locations of the equivalent number of spray units on the at least one boom.

In an example, the processing unit is configured to control the at least one first rotator actuator and/or the at least one second rotator actuator to rotate the plurality of spray units in unison by a same angle of rotation with respect to the vertical axis and/or horizontal axis.

In an example, control of the at least one second actuator by the processing unit comprises utilization of a magnitude of the determined air movement speed relative to the ground.

In an example, control of the at least one second actuator by the processing unit comprises utilization of a magnitude of an air direction angle of the determined air movement direction relative to the ground.

In an example, the air direction angle is the angle between the determined air movement direction relative to the ground and a projection of the fore-aft axis of the vehicle onto the ground.

In an example, the at least one angle of rotation with respect to the vertical axis is based at least in part on the air movement speed relative to the ground multiplied by the cosine of the air direction angle.

In an example, the at least one second actuator comprises at least one activation actuator configured to start the plurality of spray units spraying the pollen or a liquid chemical and configured to stop the plurality of spray units spraying the pollen or a liquid chemical.

In an example, the processing unit is configured to control one or more of the at least one activation actuator to stop an equivalent number of spray units from spraying the pollen or a liquid chemical based at least in part on a magnitude of the determined air movement speed relative to the ground.

In this manner, when the wind speed in combination with its direction would lead to a situation where spray from one or more spray units could cause problematic spray drift, those units can be turned off. Thus, for example a spray vehicle can operate on the margins of a field and gusting wind that would cause spray from an outer one or two spray units to cross over the border of the field can be mitigated by turning off those spray units.

In an example, the processing unit is configured to control one or more of the at least one activation actuator to stop an equivalent number of spray units from spraying the pollen or a liquid chemical based at least in part on a magnitude of an air direction angle of the determined air movement direction relative to the ground.

In an example, the air direction angle is the angle between the determined air movement direction relative to the ground and a projection of the fore-aft axis of the vehicle onto the ground

In an example, the determination to stop a spray unit from spraying is based at least in part on the air movement speed relative to the ground multiplied by the cosine of the air direction angle.

In an example, the at least one second actuator comprises at least one spray adjustment actuator configured to control a droplet size of the pollen or a liquid chemical sprayed by the plurality of spray units.

In an example, the processing unit is configured to control one or more of the at least one spray adjustment actuator to control the droplet size of the pollen or a liquid chemical sprayed by an equivalent number of spray units based at least in part on a magnitude of the determined air movement speed relative to the ground.

In an example, the processing unit is configured to increase the droplet size on the basis of an increase in the magnitude of the determined air movement speed relative to the ground.

In this manner, as the wind speed and/or direction would otherwise lead to problematic spray drift, the sprayed drop size can be increased in order to mitigate spray drift, because larger spray droplets suffer from less drift in comparison to smaller spray droplets.

In an example, the processing unit is configured to control one or more of the at least one spray adjustment actuator to control the droplet size of the pollen or a liquid chemical sprayed by an equivalent number of spray units based at least in part on a magnitude of an air direction angle of the determined air movement direction relative to the ground.

In an example, the air direction angle is the angle between the determined air movement direction relative to the ground and a projection of the fore-aft axis of the vehicle onto the ground.

In an example, the droplet size is determined at least in part on the air movement speed relative to the ground multiplied by the cosine of the air direction angle.

It would further be beneficial to mitigate the effects of downwash from a vehicle, such as an aerial vehicle, while also providing a means for interacting with an object, such as a plant.

In an example, a boom is provided which extends below the downwash of a vehicle, while also allowing the boom to come into contact with an object. The boom allows for dispersion to the object while mitigating the risk the vehicle will become entangled with the object and become damaged.

In an example, the boom is set at a length to extend beyond the downwash of the vehicle. Further, the boom is capable of traveling in both a horizontal and vertical direction in its path from the vehicle.

In an example, the vehicle is capable of an altitude measurement sensor located on the vehicle, and is capable of determining the height of the object in relation to the vehicle or the boom.

In an example, the boom can be either rigid or flexible. The boom could also have multiple components, with one part being rigid and the other being flexible. The boom could also have a rod which helps keep the boom stable in its position. The boom can also have a joint allowing the rod to bend where necessary.

In an example, the boom could have braces on the sides of the boom which act to stabilize the boom in relation to the vehicle. These braces could be made of a rigid substance, and be manipulated with actuators. The braces could also be made of wire and act to affect the boom through use of an electric wench. Sensors could also be attached at multiple locations of the boom to provide information on where the boom is in relation to the vehicle.

In an example, there is a container attached to the vehicle which contains pollen or a liquid chemical that can be transferred from the container through multiple different approaches.

In an example, there are multiple different approaches which can be used in dispensing the pollen or a liquid chemical including atomized sprays and electrostatic dispersion.

In an example, the outlet of the boom may be multiple different outlets, as compared to a single outlet.

In an example, the dispersion of pollen or a liquid chemical may occur through a lowered component hanging below the vehicle on wires. The lowered component could also have a boom attached thereto.

In an example, the liquid pollen or a liquid chemical could be dispersed using a string, with liquid running down the string to the place for dispersion. This may also be done with the string in a loop, and the wire being moved through the vehicle by a motor to increase the rate of liquid applied to the string.

In an example, the vehicle could be capable of using an imaging system to identify the objects on which to disperse the pollen or a liquid chemical. The imaging system could be able to detect a specific area of the object, such as a specific component of a plant for dispersion. The imaging system may be further aided by a system, such as a neural network, to identify the components of the object for dispensing. In an example, the system, including an imaging system, identifies a location of a plant to set a timing sequence for dispersion along a row of plants.

In an example, the movement of pollen or a liquid chemical from the container on the vehicle includes a metering system capable of measuring the amount of substance moved from the container. This measurement may be done by a number of different potential approaches.

### BRIEF DESCRIPTION OF THE DRAWINGS

Exemplary embodiments will be described in the following, by way of example only, with reference to the following drawings:

FIG. 1 shows a schematic set up of an example of a spray vehicle;

FIG. 2 shows a schematic example of a plan view of a spray vehicle;

FIG. 3 shows a schematic set up of an example of a spray units attached to a boom;

FIG. 4 shows a schematic set up of an example of a spray units attached to a boom;

FIG. 5 shows an embodiment of a spray vehicle with a boom to reduce downwash and damage;

FIG. 6 shows an embodiment where the boom transverses in a horizontal and vertical direction;

FIG. 7 shows an embodiment with a rod supporting the boom;

FIG. 8 shows a boom with two separate sections both flexible and rigid;

FIG. 9 shows an embodiment where the boom is supported by rods or wires with corresponding actuators or wenches;

FIG. 10 shows an embodiment where the boom is being moved by the actuators or wenches;

FIG. 11 shows a sprayer attached to the end of the boom;

FIG. 12 shows that an electrostatic charge be applied along the boom;

FIG. 13 shows multiple outlets at the end of the boom;

FIG. 14 shows multiple outlets extending from a central boom;

FIG. 15 shows a guided attachment at the end of the boom;

FIG. 16 shows a boom with multiple outlets containing guided attachments;

FIG. 17 shows an embodiment of the guided attachment on an object;

FIG. 18 shows an embodiment of the lowered dispersal component;

FIG. 19 shows an embodiment of the lowered dispersal component with multiple outlets;

FIG. 20 shows an embodiment of the pollen string;

FIG. 21 shows an embodiment of the pollen string as a loop;

FIG. 22 shows an embodiment of multiple pollen strings; and

FIG. 23 shows an embodiment with a measurement device and an imaging device.

### DETAILED DESCRIPTION OF EMBODIMENTS

### Boom for Spraying of Pollen or a liquid chemical to Account For Wind Drift

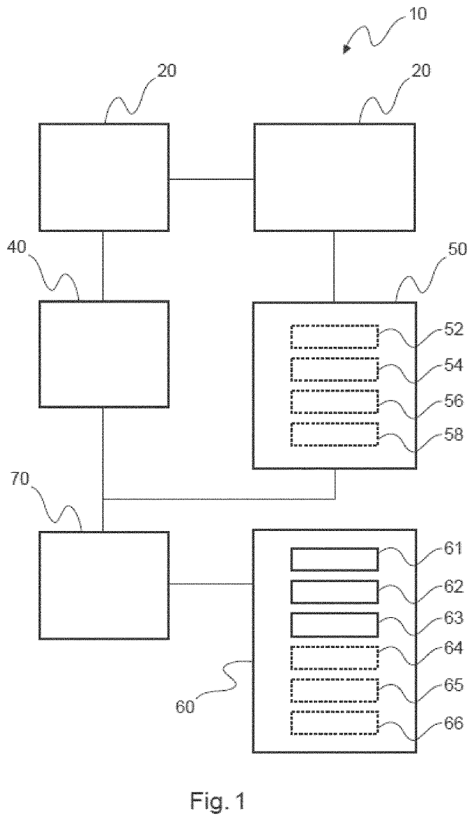

FIG. 1 shows an example of a spray vehicle 10. The spray vehicle comprises at least one boom 20, a plurality of spray units 30, at least one first actuator 40, at least one second actuator 50, a plurality of sensors 60, and a processing unit 70. The at least one boom is movably attached to the vehicle. The plurality of spray units are attached to the at least one boom. The plurality of spray units are configured to spray a pollen or a liquid chemical. The at least one first actuator is configured to move the at least one boom. The at least one second actuator is configured to control the plurality of spray units. At least one sensor 61 of the plurality of sensors is configured to measure a speed of the vehicle relative to the ground. At least one sensor 62 of the plurality of sensors is configured to measure an air movement direction relative to the vehicle. At least one sensor 63 of the plurality of sensors is configured to measure an air movement speed relative to the vehicle. The processing unit is configured to determine an air movement direction relative to the ground and determine an air movement speed relative to the ground. The determination of the air movement direction relative to the ground and the air movement speed relative to the ground comprises utilization of the speed of the vehicle, the air movement direction relative to the vehicle and the air movement speed relative to the vehicle. The processing unit is configured also to control the at least one first actuator and/or the at least one second actuator. The control of the at least one first actuator and/or of the at least one second actuator comprises utilization of the determined air movement direction relative to the ground and the determined air movement speed relative to the ground.

In an example, the at least one sensor 61 configured to measure a speed of the vehicle relative to the ground comprises a GPS system.

In an example, the at least one sensor 61 configured to measure a speed of the vehicle relative to the ground comprises a laser reflectance based system.

In an example, the at least one sensor 61 configured to measure a speed of the vehicle relative to the ground comprises a system linked to the transmission of the vehicle.

In an example, the at least one sensor 62 configured to measure an air movement direction relative to the vehicle comprises a wind vane.

In an example, the at least one sensor 63 configured to measure an air movement speed relative to the vehicle comprises an anemometer.

According to an example, the at least one first actuator is configured to move the at least one boom in a vertical direction, and the processing unit is configured to control the at least one first actuator to move the at least one boom in a vertical direction.

According to an example, at least one sensor 64 of the plurality of sensors is configured to provide data from which a height of the at least boom above the ground can be determined. The processing unit is configured to control the at least one first actuator to position the at least one boom at a height above the ground that depends on a magnitude of the determined air movement speed relative to the ground.

In an example, the sensor used to determine height is a radar sensor.

In an example, the sensor used to determine height is a laser time of flight sensor.

In an example, the sensor used to determine height is a sensor that determines the position of the boom with respect to an end stop, and assuming that the ground is flat calculates the height above the ground from the position of the boom with respect to the vehicle.

According to an example, the processing unit is configured to control the at least one first actuator to position the at least one boom at a height above the ground that depends on a magnitude of an air direction angle of the determined air movement direction relative to the ground.

According to an example, the air direction angle is the angle between the determined air movement direction relative to the ground and a projection of the fore-aft axis of the vehicle onto the ground.

According to an example, the height above the ground at which the at least one boom is positioned is calculated on the basis of the air movement speed relative to the ground multiplied by the cosine of the air direction angle.

According to an example, the processing unit is configured to control the at least one first actuator to move the at least one boom in a downwards direction when the magnitude of the determined air movement speed relative to the ground exceeds one or more threshold values.

According to an example, the one or more threshold values depend upon a magnitude of an air direction angle. The air direction angle is the angle between the determined air movement direction relative to the ground and a projection of the fore-aft axis of the vehicle onto the ground.

According to an example, the one or more threshold values are a plurality of threshold values. A threshold value of the plurality of threshold values is calculated on the basis of a set air movement speed multiplied by the cosine of the air direction angle.

According to an example, the plurality of spray units are movably attached to the at least one boom and at least one sensor 65 of the plurality of sensors is configured to provide data from which an angle of the plurality of spray units with respect to a vertical axis can be determined. The at least one second actuator 50 comprises at least one first rotator actuator 52 configured to rotate the plurality of spray units by at least one angle of rotation with respect to the vertical axis. The processing unit is configured to control one or more of the at least one first rotator actuator to rotate one or more of the plurality of spray units.

According to an example, a horizontal axis extends in a direction perpendicular to a fore-aft axis of the vehicle and extends in a direction perpendicular to the vertical axis. At least one sensor 66 of the plurality of sensors is configured to provide data from which an angle of the plurality of spray units with respect to the horizontal axis can be determined. The at least one second actuator 50 comprises at least one second rotator actuator 54 configured to rotate the plurality of spray units by at least one angle of rotation with respect to the horizontal axis. The processing unit is configured to control one or more of the at least one second rotator actuator to rotate one or more of the plurality of spray units.

According to an example, the processing unit is configured to control a plurality of first rotator actuators to hold an equivalent number of spray units simultaneously at different angles of rotation with respect to the vertical axis.

According to an example, control of the plurality of first rotator actuators depends upon the locations of the equivalent number of spray units on the at least one boom.

According to an example, the processing unit is configured to control a plurality of second rotator actuators to hold an equivalent number of spray units simultaneously at different angles of rotation with respect to the horizontal axis.

According to an example, control of the plurality of second rotator actuators depends upon the locations of the equivalent number of spray units on the at least one boom.

According to an example, the processing unit is configured to control the at least one first rotator actuator and/or the at least one second rotator actuator to rotate the plurality of spray units in unison by a same angle of rotation with respect to the vertical axis and/or horizontal axis.

According to an example, control of the at least one second actuator by the processing unit comprises utilization of a magnitude of the determined air movement speed relative to the ground.

According to an example, control of the at least one second actuator by the processing unit comprises utilization of a magnitude of an air direction angle of the determined air movement direction relative to the ground.

According to an example, the air direction angle is the angle between the determined air movement direction relative to the ground and a projection of the fore-aft axis of the vehicle onto the ground.

According to an example, the at least one angle of rotation with respect to the vertical axis is based at least in part on the air movement speed relative to the ground multiplied by the cosine of the air direction angle.

According to an example, the at least one second actuator comprises at least one activation actuator 56 configured to start the plurality of spray units spraying the pollen or a liquid chemical and configured to stop the plurality of spray units spraying the pollen or a liquid chemical.

According to an example, the processing unit is configured to control one or more of the at least one activation actuator to stop an equivalent number of spray units from spraying the pollen or a liquid chemical based at least in part on a magnitude of the determined air movement speed relative to the ground.

According to an example, the processing unit is configured to control one or more of the at least one activation actuator to stop an equivalent number of spray units from spraying the pollen or a liquid chemical based at least in part on a magnitude of an air direction angle of the determined air movement direction relative to the ground.

According to an example, the air direction angle is the angle between the determined air movement direction relative to the ground and a projection of the fore-aft axis of the vehicle onto the ground.

According to an example, the determination to stop a spray unit from spraying is based at least in part on the air movement speed relative to the ground multiplied by the cosine of the air direction angle.

According to an example, the at least one second actuator comprises at least one spray adjustment actuator 58 configured to control a droplet size of the pollen or a liquid chemical sprayed by the plurality of spray units.

According to an example, the processing unit is configured to control one or more of the at least one spray adjustment actuator to control the droplet size of the pollen or a liquid chemical sprayed by an equivalent number of spray units based at least in part on a magnitude of the determined air movement speed relative to the ground.

According to an example, the processing unit is configured to increase the droplet size on the basis of an increase in the magnitude of the determined air movement speed relative to the ground.

According to an example, the processing unit is configured to control one or more of the at least one spray adjustment actuator to control the droplet size of the pollen or a liquid chemical sprayed by an equivalent number of spray units based at least in part on a magnitude of an air direction angle of the determined air movement direction relative to the ground.

According to an example, the air direction angle is the angle between the determined air movement direction relative to the ground and a projection of the fore-aft axis of the vehicle onto the ground

According to an example, the droplet size is determined at least in part on the air movement speed relative to the ground multiplied by the cosine of the air direction angle.

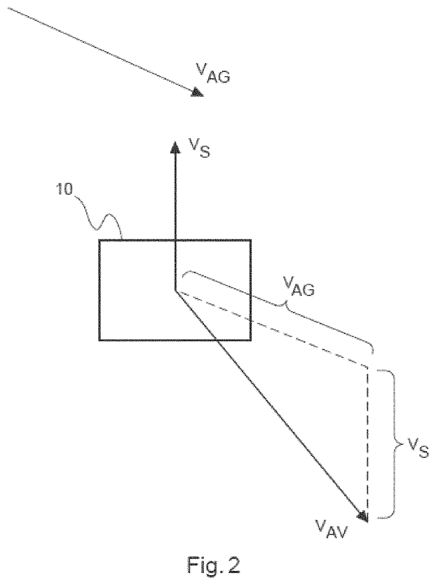

FIG. 2 shows a plan view of an exemplar vehicle 10 moving and spraying in a field, according to some embodiments. For simplicity of representation the spray booms extending either side of the vehicle are not shown. The vehicle is moving with a speed V_{S} and the wind is blowing in a particular direction and with a velocity V_{AG}. The vehicle has an anemometer and a wind vane to determine both the direction and speed of the wind, with respect to the vehicle—in other words as if the vehicle was stationary. Due to the movement of the vehicle, the measured wind direction and wind speed have both been distorted from their true ground based values, as shown. A vector based analysis can however be used to determine the real ground based wind direction and wind speed. Then, the processing unit of the vehicle can move the spray booms downwards if the wind is too strong and its direction would lead to lateral drift, and/or the processing unit can control the spray units themselves, to spray larger drops that are less susceptible to drift or angle the spray units into the wind.

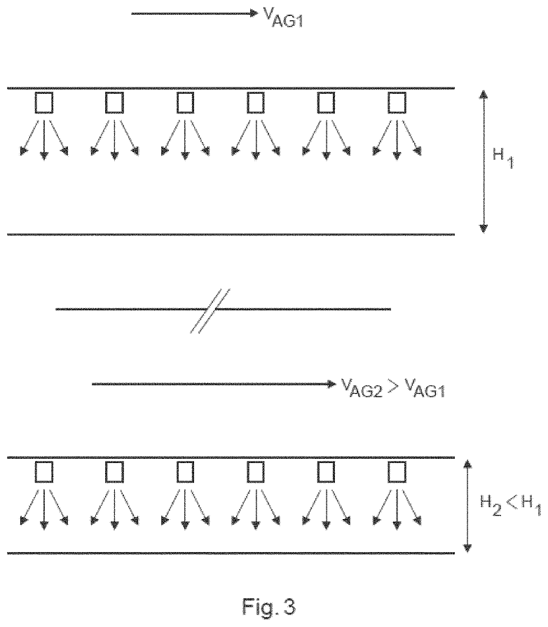

FIG. 3 schematically represents one of these situations, according to some embodiments. In the top image, the wind speed that is now in terms of a component blowing perpendicular to the vehicle's forward direction is V_{AG1}, and the processing unit has controlled appropriate actuators or actuator to hold the spray boom at a distance above ground equal to H₁, where spraying is optimized and at the same time the effects of drift are determined not to be problematic.

The wind is however gusting in terms of a wind speed varying and/or a wind direction changing, and now the component of the wind speed perpendicular to the forward direction has increased to a value V_{AG2}. In this example, the processing unit mitigates the effect of drift, that would otherwise occur, by lowering the spray boom to a height H₂, where it has been determined that there will be less drift. This is due to the spray droplets having less distance to travel before reaching the target to be sprayed and there is less drift.

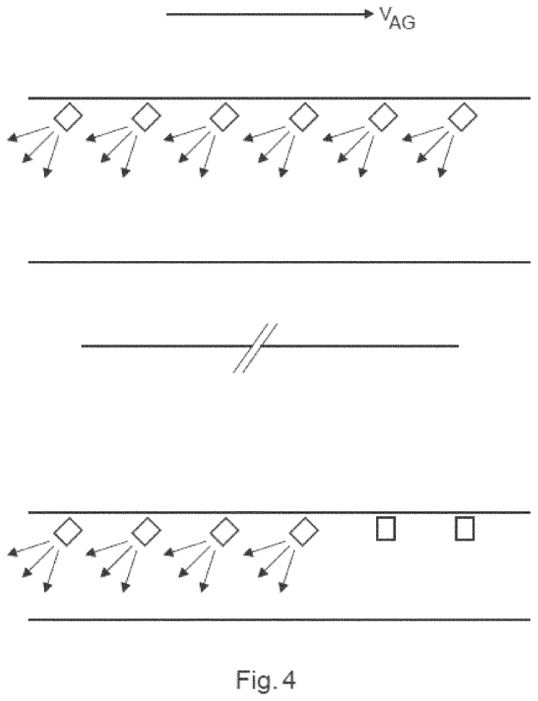

FIG. 4 schematically represents another one of these situations, according to some embodiments. In the top image, the wind speed that in terms of a component blowing perpendicular to the vehicle's forward direction is V_{AG}, and the processing unit has controlled appropriate actuators or actuator to hold the spray units at an angle into the wind. The spray then performs an arc moving upwind and then being blown downwind, with the overall sideways drift being less than would have occurred if the spray units had just sprayed downwards. In the bottom image, the vehicle is spraying on the border of a field, and the cross wind component is again as shown in the top image. Representative spray units are shown, with the left most spray unit shown, some inboard of that unit and the two right most spray units closest to the field boundary are shown. The processing unit has determined that there is no issue relating to spray drift from the left most spray unit, which consequently has been controlled to spray downwards. Moving inboard, the processing unit has controlled appropriate actuators such that different spray units are gradually angled by increasing angles to spray more into the wind. A number of spray units are shown angled by the same amount, but each can be angled by a different amount. Additionally, the processing unit has determined that due to the specific magnitude of the cross wind speed at this time there is a risk of spray from the two spray units on the right hand end of the boom crossing over the field boundary and the processing unit has controlled actuators to turn these spray units off.

It is to be noted, that the situations represented in FIGS. 3-4 can occur simultaneously, in that the boom can be raised or lowered as required and at the same time spray units angles into the wind and if necessary turned off. Also, actuators can be used to change the droplet size, thereby the processing unit can increase the drop size of the spray to mitigate drift as the wind speed increases and/or the wind direction changes to a direction where drift could become problematic.

### Boom for Spraying of Pollen or a Liquid Chemical to Reduce Downwash or Damage

It should be noted that the embodiments designed to reduce downwash or damage to a vehicle which is specifically designed with the ability to fly, and potentially using rotors to achieve flight. These embodiments should not be limited to be seen as one where only rotors are described, and should be applicable to any vehicle, as other forms of transportation can cause a downward force of air movement by their operation.

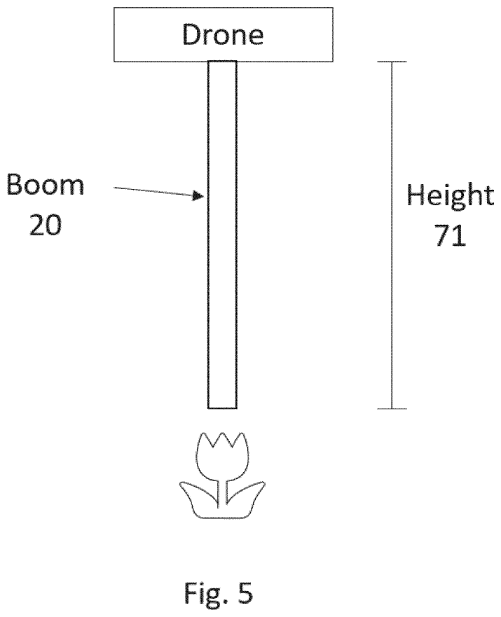

In the following embodiments, pollen or a liquid chemical could be distributed downward from a container through a boom 20, in FIG. 5, to a plant without being affected by the downward force of the vehicle's rotors, also called the downwash. This boom 20 could also provide the benefit of allowing the vehicle to provide dispersion directly onto a select portion of the plant while reducing the risk of the vehicle, and its rotors, interacting with the plants. For example, the distribution of pollen or a liquid chemical can be benefited by its delivery onto a small infected area or a pollen receiving area in concentrated amounts. Making a specific delivery onto a plant runs the risk of the vehicle interacting with the leaves, stems, and other articles of the plant that hang down or point upward from unexpected angles to impede the vehicle's movement. For example, the sensitive nature of a vehicle in flight would make such interaction difficult as it could lead to damage or stoppage of an operation spraying or dispersing to a field.

The boom would optimally be set at a length 71 to sufficiently spray without being affected by the downward force of air caused by the rotors of the vehicle. This length can be such that the downward force of air from the rotors is sufficiently diminished so that the air does not disturb the movement of pollen or a liquid chemical as it exits the boom. The length 71 of the boom could also be set at a shorter length where the downward force would not substantially impair the dispersal of pollen or a liquid chemical from the boom, this reduced length might also be done where a small amount of downward air from the vehicle rotors is used to optimize the spray.

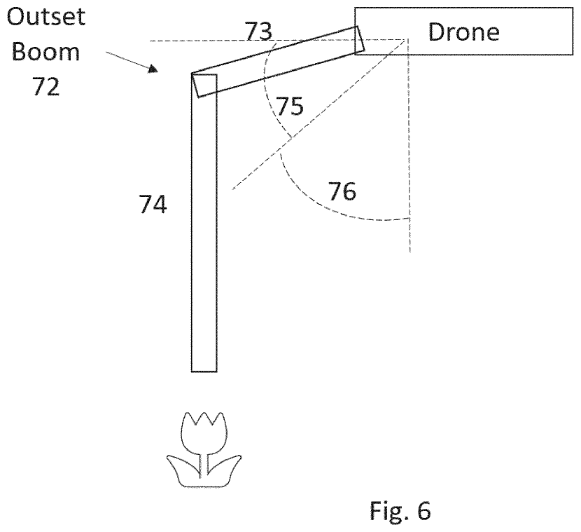

In one embodiment, as noted with drift above, an outset boom 72 is set to transverse in two directions, one direction being away from the vehicle in a horizontal axis 73, and another direction away from the vehicle in a vertical axis 74, as shown in FIG. 6. In an ideal form of this embodiment, the travel of the boom in the horizontal or vertical direction would allow spray or dispersal without being affected by the downwash of the vehicle. The horizontal direction of the boom could be any amount in the range 75 from a direct 45 degrees (45°) below the vehicle until directly parallel with the horizontal axis of the vehicle. Further, the vertical direction of the boom could be any amount in the range 76 from directly 90 degrees below the vehicle until within 45 degrees (45°) between the vertical and the horizontal axis. The boom positions could also be reversed, with the boom traversing in a vertical direction and then outward in a horizontal direction. In another embodiment, the outset boom 72 can be a in a curved manner from the vehicle along the horizontal and vertical axes.

In one embodiment, the movement of the boom 20 in the vertical axis can be set in relation to the readings of an altimeter measuring the height of the vehicle. This height could also be set by a camera, LIDAR, or series of cameras, making determinations on the believed height of the vehicle in relation to the height of the plants for pollen or a liquid chemical spray or dispersion.

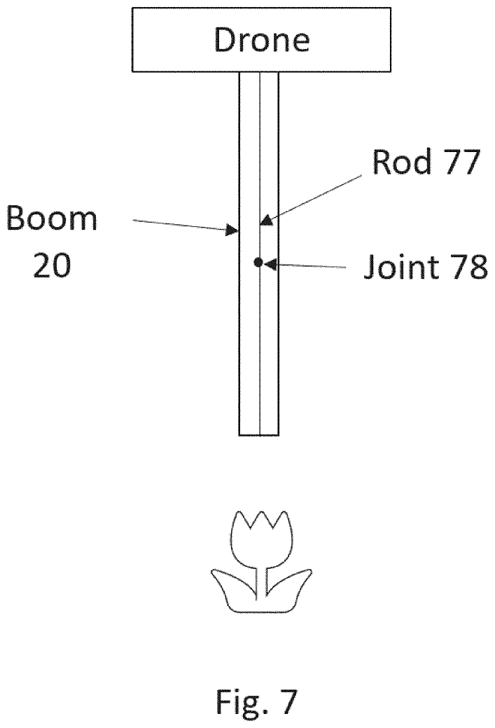

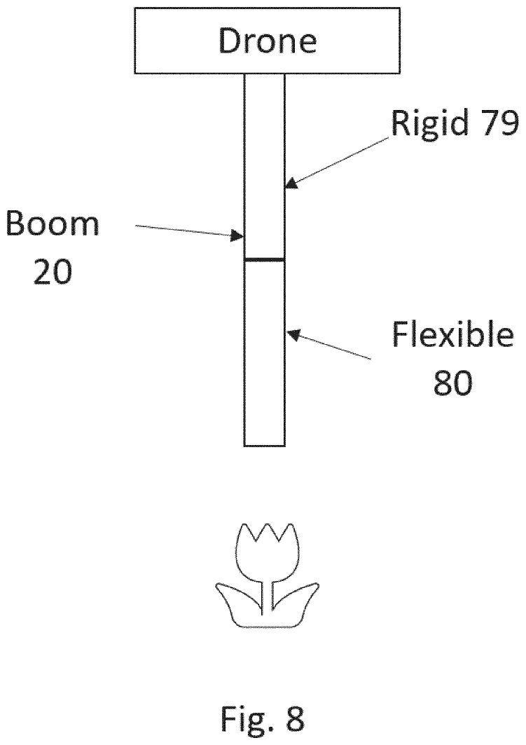

The boom 20 could either be made of a rigid or flexible material. In an ideal embodiment, the material would not prevent the flow of pollen or a liquid chemical through the boom due to being bent, kinked, or impeded by non-smooth walls. The material should also be light weight so that there is reduced overall weight of the system. In one embodiment, the boom would be made of multiple segments, capable of sliding one inside of another, to extend or reduce the length of the tubing. In another embodiment, the boom would be made of a material that could be rolled or folded to extend or reduce the length of the boom. An example would be flexible air ventilation tubing which can be shortened or extended in the same manner as accordion tubing. Flexible tubing would provide a benefit, in that it would allow the vehicle to provide an outlet near the plant with a reduced risk of impeding the vehicle's movements, as the tubing can be able to contact the plant without translating the force back to the vehicle, or at least reduce the direct force applied to the vehicle. In one embodiment, the boom 20 could be made of a flexible material, and have a rigid component, such as a rod 77 or staff, capable of keeping the boom 20 relatively stable during flight, shown in FIG. 7. This rigid component could also have a joint 78 upon which it could bend or flex thereby allowing the boom to avoid disrupting the path of flight of the vehicle when in contact with a plant. In another embodiment, the rod 77 could be able to travel along the length of the boom 20, extending along the boom 20 to provide variable stability in different situations. In another embodiment, the boom could have a rigid component where an upper component of the boom is rigid 79, whereas the lower component of the boom is flexible 80, shown in FIG. 8. This could be potentially useful where the upper component of the tubing allows the boom to maintain stability and placement below the vehicle, while the flexible component of the tubing could interact with the plant and provide high specificity on the location of delivery without the risk of providing a transverse force to the vehicle.

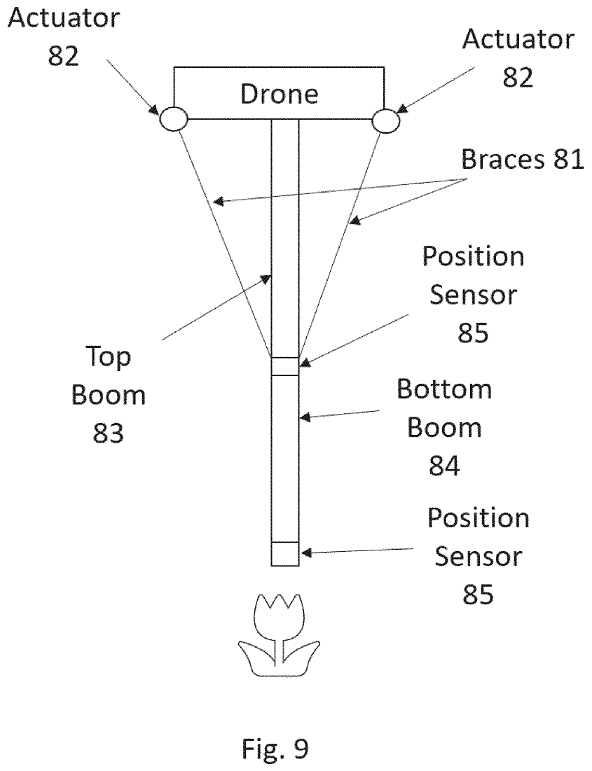

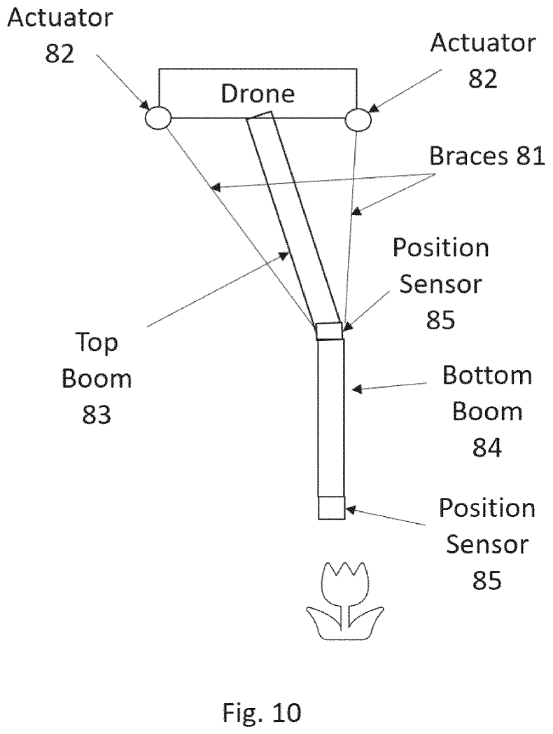

In one embodiment, the boom 20 is maintained in a position relative to the vehicle through one or more braces 81 which connect to the boom from the vehicle, shown in FIG. 9. In a preferred embodiment, the braces 81 would attach to the boom 20 at an angle to the boom 20, for example, where the braces 81 are positioned at the outer edge of the vehicle. The boom 20 could additionally be positioned in a direction by an actuator or actuators 82 which connect to the boom 20 or to the braces 81. The actuators 82 could also act to direct the boom 20 in a direction away from the vehicle to a source, such as a plant, shown in FIG. 10. Where the boom 20 is moved via braces 81, the boom 20 could have a sliding ring (not shown) on the boom 20 which allows the braces to be rigid. The braces 81 could also be extended by hydraulics potentially removing a need for a sliding ring. In one embodiment, the component moving the boom could involve wires instead of braces. The wires could be extended and retracted by the movement of an electric wench, with one side retracting and the other side extending. In one embodiment, the control of the boom 20 around the vehicle by braces 81 or wires would consist of at least three (3) braces or wires with corresponding actuators or electric wenches. In a preferred embodiment, control around the vehicle would be done through four (4) braces 81 or wires with corresponding actuators or electric wenches. In this embodiment, the braces could extend to the lower component of the boom 20, or could extend to an intermediate area of the boom 20. The boom 20 could consist of a top portion 83 and a bottom portion 84. The top boom 83 could be able to articulate in a direction different from the bottom boom 84. Further, as previously noted, the bottom boom 84 could be made of a flexible material. In an embodiment, the position of the boom 20 in relation to the vehicle may be monitored through a position sensor 85 or sensors on the boom 20. In a preferred embodiment, the position of the boom 20 in relation to the vehicle could be noted by a position sensor 85 at the point the braces 81 or wires connect with the boom 20. In another preferred embodiment, the sensors 85 could be located at the point where the top boom 83 and bottom boom 84 meet, and another sensor 85 where the bottom boom 84 disperses to the source or plant.

The boom can transfer pollen or a liquid chemical from a storage container through use of gravity, an auger, a fan, compressed air, a belt, or through a sieve (not shown). The boom could also simply transfer the substance down the boom through the movement of the vehicle which could allow the substance to be moved downward in the same manner as being shaken out of a boom. In this manner, the vehicle could be flown in a pattern to cause the boom to move or shake pollen or a liquid chemical from the container and through the boom, for example, where it is flown in a specific pattern, such as a circular pattern. The pollen or a liquid chemical could be metered into the boom by means of a sieve, valve, auger, or other metering system to allow only a portioned amount into the boom. The lower portion of the boom could also have a valve which meters the dispersal from the boom.

The boom could be of a variety of widths in overall diameter of the boom, as well as inner diameter and outer diameter of the boom itself. In embodiments using dry particulate substance, the boom would need to have a width sufficient to allow the flow of dry particulate substance through the boom.

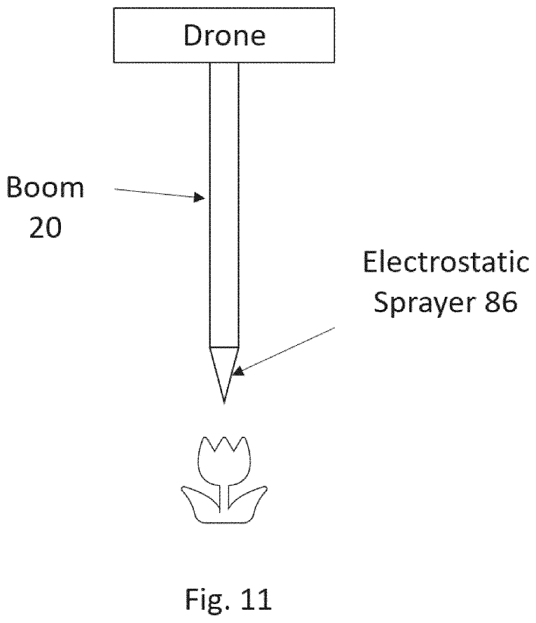

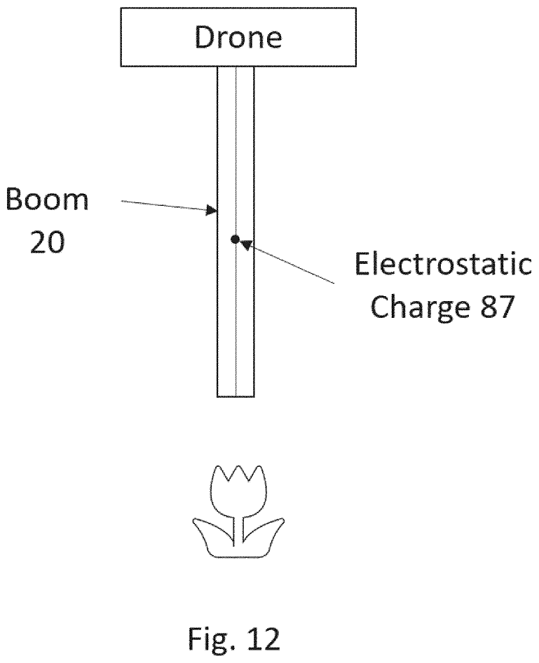

Dispersal can also be done in conjunction with an electrostatic component. This could be in the form of an electrostatic sprayer 86 at the lower end of the boom, as in FIG. 11. It could be in the form of a component providing an electrostatic charge 87 prior to being sprayed, such as at the outlet of the storage prior to being transported to the boom, as in FIG. 12. The electrostatic charge potential difference can facilitate a net attraction between the substance and the plants, for example, between the pollen grains and the plant. The electrostatic charge could be applied as the pollen or a liquid chemical exit the boom or directly after the storage outlet to prevent adherence of substance to boom inner walls.

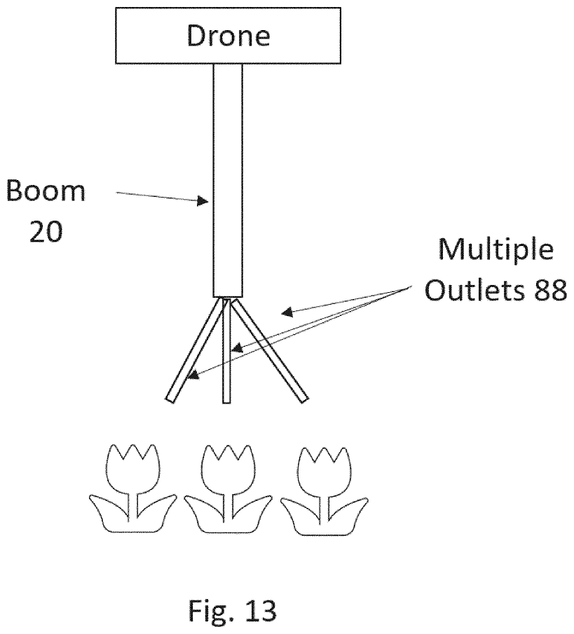

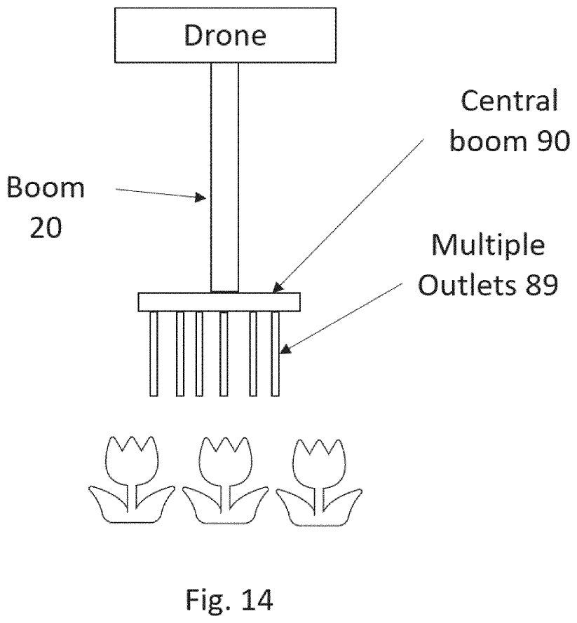

The boom 20 can also, in one embodiment, have multiple outlets 88 at its base, as in FIG. 13. These multiple outlets 88 could be placed in such a manner to allow spraying different areas of a single plant, or capable of spraying multiple plants at one time. Multiple nozzles could be used to broadcast pollen or a liquid chemical in a spherical pattern to accommodate the random nature of the silks. In another embodiment, the vehicle will have a boom 20 with outlets 89 available at sections around the center of the lower portion of the vehicle, for example there could be six (6) outlets, as in FIG. 14. These outlets stem from a central boom 90 where pollen or a liquid chemical is provided. The boom 20 could have a component capable of providing pollen or a liquid chemical to any of the multiple different areas around the vehicle. For example, if the boom has outlets which, when seen from a top down view of the vehicle, provide an outlet in a range around the vehicle, then the vehicle can orient itself over the plant, with the ability to distribute pollen or a liquid chemical through the boom to any point around the plant. This can be beneficial as it would allow the dispersion of pollen or a liquid chemical around the plant without requiring any form of direction control on the end of the boom. Instead, an end of the boom would be available and directable to a receiving component of the plant.

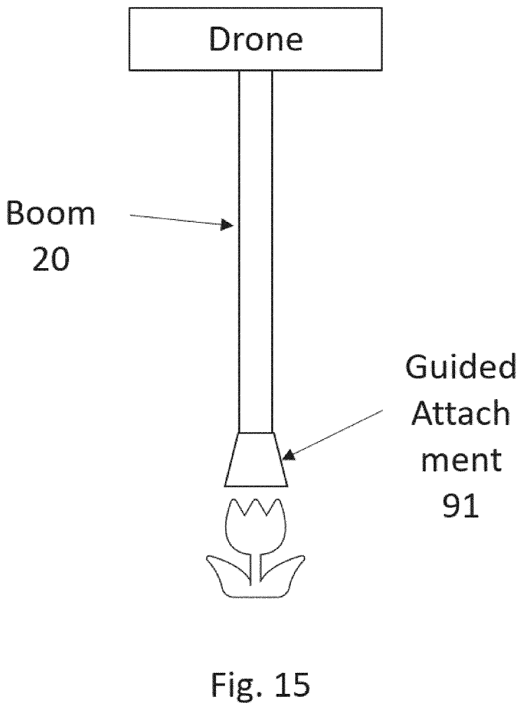

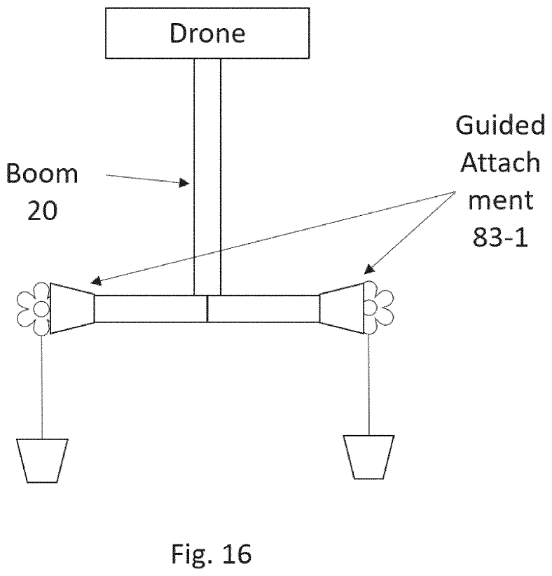

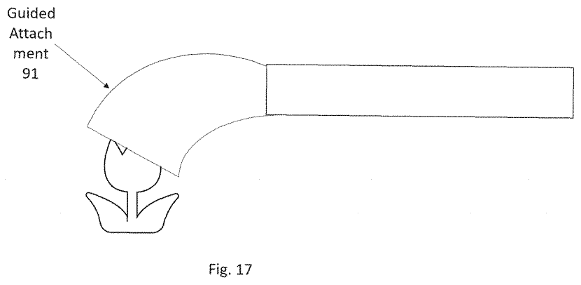

Furthermore, in an embodiment, the boom 20 could use a guided attachment 91 as part of the dispersal, shown in FIG. 15. This guided attachment could be in a cone shape capable of surrounding the area on which to disperse. This attachment can be placed on the object and would be sprayed to ensure a dose of liquid received only to that portion of the object. The guided attachment could also include a sensor (not shown) capable of signifying when the object on which to be dispersed is in position under the guided attachment 91. The boom 20 could be set in such a manner that it extends vertically downward and then horizontally outward to reach the correct placement on the object to be dispersed, as in FIG. 16. The guided attachment could then be used on the horizontal component of the boom to disperse to the object. The guided attachment could be in a variety of different shapes, such that the object on which to be dispersed is captured in the guided attachment, see FIG. 17 for a preferred embodiment of a guided attachment 91 design with a curved body to encapsulate the object and provide a condensed dispersal to the object.

### Lowered Dispersal Component

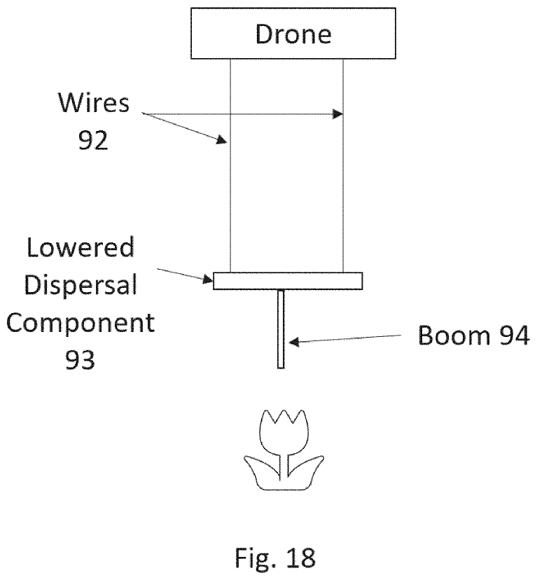

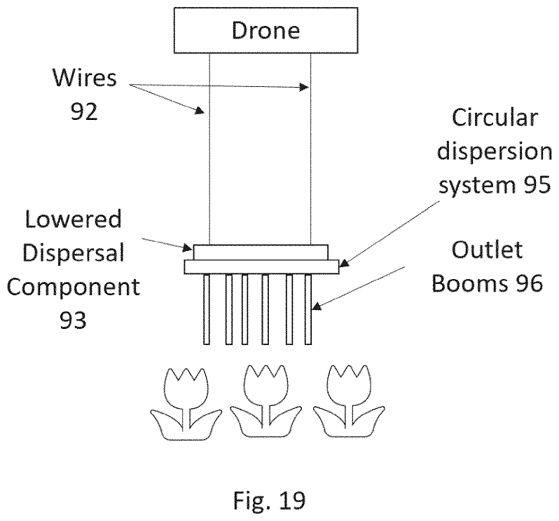

In one embodiment, the dispersal component of the vehicle could be a separate system which is maintained at a distance below the vehicle, as in FIG. 18. This component could be connected to the vehicle via wires or cables 92 which allow the dispersal component to be below the downwash of the vehicle, for the reasons as noted above. This lowered dispersal component 93 can include both the dispersal system as well as a container. The lowered dispersal component could be designed with dispersal in the form of a boom 94 extending from the lowered dispersal component 93. The dispersal component could also be in other forms, such as a spray nozzle, including, but not limited to, electrostatic sprayer, or in any other form of dispersion. An example would simply be a central dispersion system 95, which could disperse a substance to multiple outlet booms 96, shown in FIG. 19. In one embodiment, the central dispersion system 95 could disperse along a circular path around the vehicle, where the central dispersion system 95 is circular in shape. In this embodiment, the vehicle could become centered above a plant, and the pollen or a liquid chemical would be dispersed to either a specific part of the circle, or could be dispersed around the entire circle, or could also be dispersed throughout the entire interior area of the circle. The dispersion could be done by simply being deposited from the central dispersion system 95, or the dispersion could occur through the outlet booms 96. It should be noted this may be done with any number of shapes for the central dispersion system 95. In one embodiment, pollen or a liquid chemical is distributed in the form of a granular ring (not shown). This can be accomplished in the form of a dispersion component, similar to granular rings for dispersal of seeds or other material relating to agriculture or lawn care. Another embodiment of a dispersion system from the lowered dispersal component 93 could involve a spray powder in a gas stream, either of air or nitrogen. In another embodiment, the lowered portion of the vehicle could disperse the pollen or a liquid chemical through a separate propeller, or a small turbine capable of sending pollen or a liquid chemical in a specific direction.

In a preferred embodiment, the lowered dispersal component could have the ability to determine its height from the ground in the same manner as described throughout this application. The vehicle could optionally have a similar height measurement system to provide a means to provide a more precise height level of the vehicle and the lowered component. The lowered dispersal component 93 would be able to communicate with the vehicle, and the vehicle could provide power to the lowered component via the wires 92. One benefit of the lowered dispersal component 93 would be the ability to more quickly take pollen or a liquid chemical from the container to the plant, as compared to travelling through a boom 20.

### Liquid Pollen or a Liquid Chemical Dispensing

Where the vehicle is dispersing pollen in a liquid form or a chemical, the vehicle can have specific components to facilitate delivery of the liquid. The liquid can be applied to the plants in a stream or atomized spray, using any permeation of a spray nozzle. The liquid delivery system can be done by any of the aforementioned methods for a boom 20. In an embodiment dispersing only a liquid, the boom 20 would likely be able to be of a much smaller width, capable of dispersing liquid without causing the boom 20 to become blocked or impede the flow of the liquid.

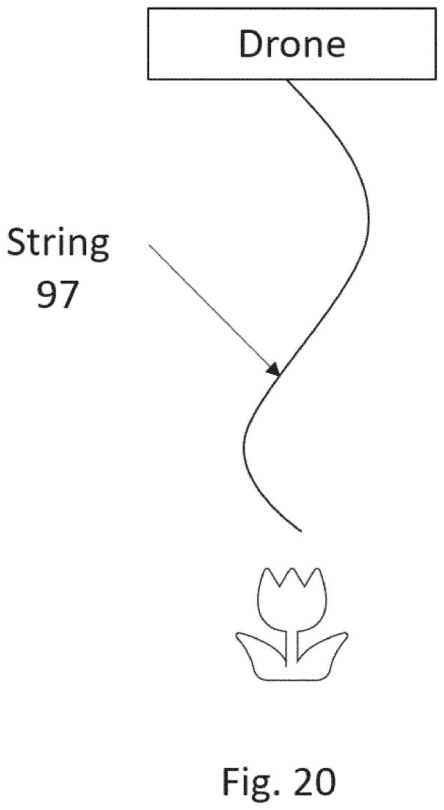

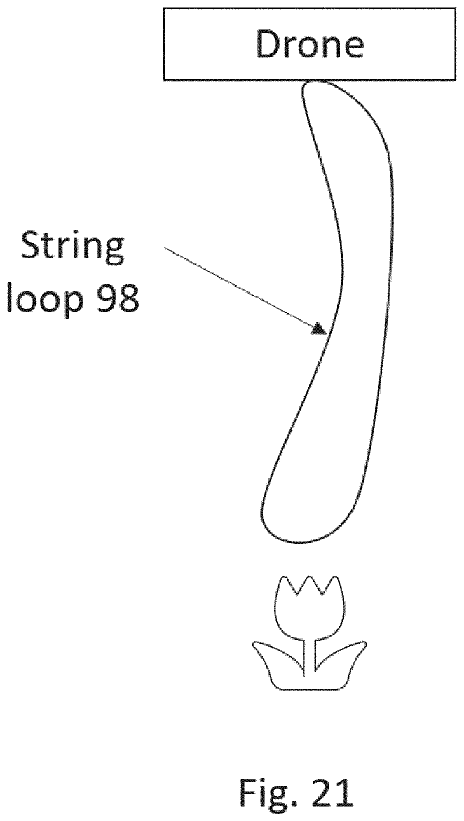

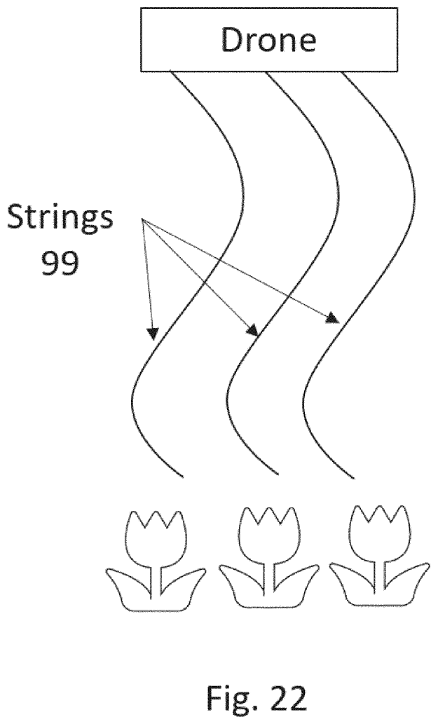

In one embodiment, the liquid could be distributed by being deposited onto a string 97 hanging below the vehicle, shown in FIG. 20. This string 97 could be made of a strong or durable material such as Teflon. This string 97 would allow the liquid to slowly descend the length of the string 97 transferring the liquid from a container to the end of the string 97. At the end of the string 97, the liquid could be directly applied to a plant, or could detach from the string 97 and drop onto the plant through the force of gravity. The string 97 dispersal approach could also be accomplished from the action of a string 97 running in a loop 98, shown in FIG. 21, or being connected on a dragline (not shown). This loop 98 or dragline would be able to move in a circular fashion, allowing the string to cycle through an area where it could come into contact with liquid in a storage container, and then later be brought to a lower point along its path thereby coming into contact with the plant. There could also be multiple different strings 99, shown in FIG. 22) from a given vehicle, allowing greater probability of making contact with a plant while reducing the need for specific targeting or without requiring any targeting of the pollen receiving component. Multiple strings 99 could also be used to apply to multiple plants. Though not shown, there may also be multiple loops 98.

### Pollen or a Liquid Chemical Dispersal on an Imaging Basis and a Timing Basis

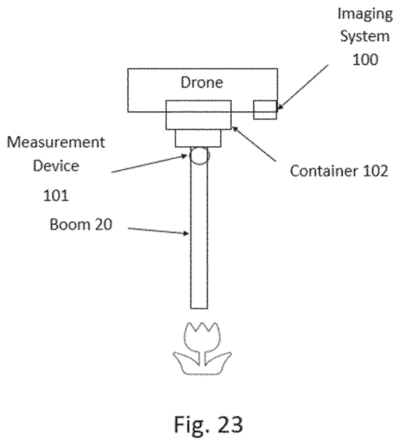

The dispersal of the pollen or a liquid chemical can be based on the use of a sensor to identify a specific plant. This can be done by a camera or imaging system 100 which can identify the plant from above the vehicle, shown in FIG. 23. This can also be done through use of a spectral signature associated with a specific type of plant, or a specific area of a plant. Once the plant or component of the plant is identified, pollen or a liquid chemical could then be dispersed to the plant. This dispersal would, in a preferred embodiment, be done in such a way that the pollen or a liquid chemical is dispersed in an area on the targeted plant. It should be noted, this system would ideally target the plant by examining the entire plant, though it could also locate a specific piece or component of a plant. Further analysis of the plant can be made by software related to the imaging system 100, which could provide a best estimate for the likely radius within which the specific area for targeting on the plant would be located. The imaging system would adjust for height and angle of dispersal. This estimated radius, could then be used to adjust the amount of pollen or a liquid chemical to be dispersed and the amount metered through the vehicle, and the amount of force used in dispersing from the boom or dispensing device, thus increasing or decreasing the radius of the dispersal area.

In addition, the ability to determine a specific area of a plant for dispersal can be enhanced through use of a system, such as deep learning using an artificial neural network. This could use images showing the plant, and the specific areas of the plants for targeting. These images could be used to train the artificial neural network to improve the ability to target specific areas on the plant.

In another embodiment, pollen or a liquid chemical can be dispersed in a direction, or in a line, with the dispersal being completed on a continuous flow, or through metered amounts based on a timing sequence. Where based on a timing sequence, the dispersal could be based on the known distance between each plant. For example, plants might be planted in a row 30 inches apart. An algorithm could be made to account for the speed of the vehicle, and to be set for a dispersal at such a time where it would then disperse on each plant. In another embodiment, the dispersal could be performed on a combination of a sensor to detect a plant with a timing sequence for dispersal. The sensor would be able to align the vehicle upon a plant and operate in the direction of the next plant, such as a crop line, dispensing based on a timing sequence, while also using the sensing mechanism to denote where the vehicle is failing to disperse to the correct area.

### Measurement Device

In one embodiment, the pollen or a liquid chemical being dispensed is monitored through a measurement or metering device 101, shown in FIG. 23. The measurement device could be used to measure the flow of substance from the container 102 through the boom 20. This measurement device 95 can be performed by use of an auger, or through use of a flow rate sensor, or an imaging device capable of measuring the size of particles through optical character recognition. This also can be done through the use of a scale capable of measuring the weight of a particular section of the tubing or an output portion of the tubing.

The measurement device would be able to provide feedback on the amount of pollen or a liquid chemical being dispensed as the vehicle is being used. This information can be transmitted to an external computer, or stored on the vehicle to be delivered to an external computer at a later time. In one embodiment, the measurement device would be able to provide the grams per second being delivered for dispersal. In another embodiment, the measurement device output would be connected to a software system which can take the measurements and output them into a system which can be used for further analysis of the dispensed system.

### SUMMARY OF INVENTIONS

According to some embodiments, the invention includes: a vehicle, comprising: a container capable of carrying a substance and at least one boom capable of dispersing the substance to a receiving object, with the boom being at such a length to extend beyond the downward force of air caused by the vehicle. The vehicle according to this embodiment, wherein the as least one boom is adjustable in the vertical direction. The vehicle according to this embodiment, wherein the at least one boom is capable of extending and contracting away from the vehicle. The vehicle according to this embodiment, where the boom is made of a material capable of folding or rolling to allow for the extension or contraction of the boom. The vehicle according to this embodiment, wherein the extension and contraction of the boom is performed by multiple segments of the at least one boom which slide in relation to each other. The vehicle according to this embodiment, wherein the multiple segments of the at least one boom are made of a rigid material. The vehicle according to this embodiment, wherein the at least one boom consists of rigid and flexible components. The vehicle according to this embodiment, wherein the at least one boom is of a flexible material with a rod affixed to the at least one boom providing stability to all or a portion of the flexible boom. The vehicle according to this embodiment, wherein the at least one boom is of a flexible material a rod able to travel along the length of the boom providing stability. The vehicle according to this embodiment, wherein the at least one boom is of a flexible material and a rod extends along the boom to provide stability and the rod has a joint allowing the rod to bend when pressure is applied to the rod. The vehicle according to this embodiment, wherein the at least one boom is a rigid material closer to the vehicle and a flexible material near the location where the at least one boom will interact with the object. The vehicle according to this embodiment, wherein the at least one boom travels in both a horizontal and vertical direction. The vehicle according to this embodiment, wherein a sensor is used to measure the height of the vehicle in relation to the at least one boom. The vehicle according to this embodiment, wherein the at least one boom is positioned relative to the vehicle by at least one brace attached to the outer edge of the drone, wherein the at least one brace is able to be manipulated through actuators to control the movement of the at least one boom. The vehicle according to this embodiment, wherein the at least one boom is positioned relative to the vehicle through at least one wire attached to the outer edge of the drone, wherein the at least one wire is manipulated through an electric wench located at the drone to control the movement of the at least one boom. The vehicle according to this embodiment, wherein the at least one boom has at least one sensor on the boom providing location data to the vehicle. The vehicle according to this embodiment, wherein the at least one boom has multiple exits to disperse the substance to multiple locations. The vehicle according to this embodiment, wherein the end of the at least one boom includes a guided attachment. The vehicle according to this embodiment, wherein the dispersal from the at least one boom can be targeted onto a specific area of a receiving object through a targeting system, wherein the targeting system is operated through use of a neural network. The vehicle according to this embodiment, wherein the dispersal from the at least one boom can be targeted onto an entire object though use of a system based on targeting a plant from a row of plants and a timing sequence to disperse to each plant in said row of plants. The vehicle according to this embodiment, wherein a measurement device is at the exit of the container and could take a measurement of the amount of substance moving out of the container over time.

According to some embodiments, the invention includes: a vehicle, comprising: lowered dispersal component including the container and capable of being connected to the vehicle at a distance below the drone such that it would avoid the downward force of air caused by the vehicle. The vehicle according to this embodiment, wherein at least one boom extend below the lowered dispersal component. The vehicle according to this embodiment, wherein the lowered dispersal component has sensors capable of determining the height of the lowered dispersal component and the ground.

According to some embodiments, the invention includes: a vehicle, comprising: a container capable of carrying a substance and at least one string capable of dispersing liquid and able to descend below the vehicle. The vehicle according to this embodiment, wherein the string is in the shape of a loop.

While embodiments of the invention has been illustrated and described in detail in the exemplary drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A spray vehicle, comprising:
at least one boom;
a plurality of spray units;
at least one first actuator;
at least one second actuator;
a plurality of sensors; and
a processing unit;
wherein, the at least one boom is movably attached to the vehicle;
wherein, the plurality of spray units are attached to the at least one boom;
herein, the plurality of spray units are configured to spray pollen or a liquid chemical;
wherein, the at least one first actuator is configured to move the at least one boom;
wherein, the at least one second actuator is configured to control the plurality of spray units;
wherein, at least one sensor of the plurality of sensors is configured to measure a speed of the vehicle relative to the ground;
wherein, at least one first sensor of the plurality of sensors is configured to measure an air movement direction relative to the vehicle;
wherein, at least one second sensor of the plurality of sensors is configured to measure an air movement speed relative to the vehicle;
wherein, the processing unit is configured to determine an air movement direction relative to the ground and determine an air movement speed relative to the ground, the determination comprising utilization of the speed of the vehicle, the air movement direction relative to the vehicle and the air movement speed relative to the vehicle; and
wherein, the processing unit is configured to control the at least one first actuator and/or the at least one second actuator; the control comprising utilization of the determined air movement direction relative to the ground and the determined air movement speed relative to the ground.

2. The spray vehicle of claim 1, wherein the at least one first actuator is configured to move the at least one boom in a vertical direction, and wherein the processing unit is configured to control the at least one first actuator to move the at least one boom in the vertical direction.

3. The spray vehicle of claim 2, wherein at least one third sensor of the plurality of sensors is configured to provide data from which a height of the at least one boom above the ground can be determined, and wherein the processing unit is configured to control the at least one first actuator to position the at least one boom at a height above the ground that depends on a magnitude of the determined air movement speed relative to the ground.

4. The spray vehicle of claim 3, wherein the processing unit is configured to control the at least one first actuator to position the at least one boom at a height above the ground that depends on a magnitude of an air direction angle of the determined air movement direction relative to the ground.

5. The spray vehicle of claim 4, wherein the air direction angle is the angle between the determined air movement direction relative to the ground and a projection of a fore-aft axis of the vehicle onto the ground.

6. The spray vehicle of claim 5, wherein the height above the ground at which the at least one boom is positioned is calculated based on the air movement speed relative to the ground multiplied by cosine of the air direction angle.

7. The spray vehicle of claim 3, wherein the processing unit is configured to control the at least one first actuator to move the at least one boom in a downwards direction when the magnitude of the determined air movement speed relative to the ground exceeds one or more threshold values.

8. The sprayer vehicle of claim 7, wherein the one or more threshold values depend upon a magnitude of an air direction angle, wherein the air direction angle is the angle between the determined air movement direction relative to the ground and a projection of a fore-aft axis of the vehicle onto the ground.

9. The spray vehicle of claim 8, wherein the one or more threshold values are a plurality of threshold values, and wherein a threshold value of the plurality of threshold values is calculated based on a set air movement speed multiplied by cosine of the air direction angle.

10. The spray vehicle of claim 1, wherein the plurality of spray units are movably attached to the at least one boom and wherein at least one fourth sensor of the plurality of sensors is configured to provide data from which an angle of the plurality of spray units with respect to a vertical axis can be determined; and wherein the at least one second actuator comprises at least one first rotator actuator configured to rotate the plurality of spray units by at least one angle of rotation with respect to the vertical axis, and wherein the processing unit is configured to control one or more of the at least one first rotator actuator to rotate one or more of the plurality of spray units.

11. The spray vehicle of claim 10, wherein a horizontal axis extends in a direction perpendicular to a fore-aft axis of the vehicle and extends in a direction perpendicular to the vertical axis; wherein at least one fifth sensor of the plurality of sensors is configured to provide data from which an angle of the plurality of spray units with respect to the horizontal axis can be determined; and wherein the at least one second actuator comprises at least one second rotator actuator configured to rotate the plurality of spray units by at least one angle of rotation with respect to the horizontal axis, and wherein the processing unit is configured to control one or more of the at least one second rotator actuator to rotate one or more of the plurality of spray units.

12. The spray vehicle of claim 11, wherein the processing unit is configured to control a plurality of first rotator actuators to hold an equivalent number of spray units simultaneously at different angles of rotation with respect to the vertical axis.

13. The spray vehicle of claim 12, wherein control of the plurality of first rotator actuators depends upon the locations of the equivalent number of spray units on the at least one boom.

14. The spray vehicle of claim 11, wherein the processing unit is configured to control a plurality of second rotator actuators to hold an equivalent number of spray units simultaneously at different angles of rotation with respect to the horizontal axis.

15. The spray vehicle of claim 14, wherein control of the plurality of second rotator actuators depends upon the locations of the equivalent number of spray units on the at least one boom.

16. The spray vehicle of claim 11, wherein the processing unit is configured to control the at least one first rotator actuator and/or the at least one second rotator actuator to rotate the plurality of spray units in unison by a same angle of rotation with respect to the vertical axis and/or horizontal axis.

17. The spray vehicle of claim 11, wherein control of the at least one second actuator by the processing unit comprises utilization of a magnitude of the determined air movement speed relative to the ground.

18. The spray vehicle of claim 11, wherein control of the at least one second actuator by the processing unit comprises utilization of a magnitude of an air direction angle of the determined air movement direction relative to the ground.

19. The spray vehicle of claim 18, wherein the air direction angle is the angle between the determined air movement direction relative to the ground and a projection of the fore-aft axis of the vehicle onto the ground.

20. The spray vehicle of claim 19, wherein the at least one angle of rotation with respect to the vertical axis is based at least in part on the air movement speed relative to the ground multiplied by the cosine of the air direction angle.

21. The spray vehicle of claim 1, wherein the at least one second actuator comprises at least one activation actuator configured to start the plurality of spray units spraying the pollen or a liquid chemical and configured to stop the plurality of spray units spraying the pollen or a liquid chemical.

22. The spray vehicle of claim 21, wherein the processing unit is configured to control one or more of the at least one activation actuator to stop an equivalent number of spray units from spraying the pollen or a liquid chemical based at least in part on a magnitude of the determined air movement speed relative to the ground.

23. The spray vehicle of claim 21, wherein the processing unit is configured to control one or more of the at least one activation actuator to stop an equivalent number of spray units from spraying the pollen or a liquid chemical based at least in part on a magnitude of an air direction angle of the determined air movement direction relative to the ground.

24. The spray vehicle of claim 23, wherein the air direction angle is the angle between the determined air movement direction relative to the ground and a projection of a fore-aft axis of the vehicle onto the ground.

25. The spray vehicle of claim 24, wherein the determination to stop a spray unit from spraying is based at least in part on the air movement speed relative to the ground multiplied by cosine of the air direction angle.

26. The spray vehicle of claim 1, wherein the at least one second actuator comprises at least one spray adjustment actuator configured to control a droplet size of the pollen or a liquid chemical sprayed by the plurality of spray units.

27. The spray vehicle of claim 26, wherein the processing unit is configured to control one or more of the at least one spray adjustment actuator to control the droplet size of the pollen or a liquid chemical sprayed by an equivalent number of spray units based at least in part on a magnitude of the determined air movement speed relative to the ground.

28. The spray vehicle of claim 27, wherein the processing unit is configured to increase the droplet size on the basis of an increase in the magnitude of the determined air movement speed relative to the ground.

29. The spray vehicle of claim 26, wherein the processing unit is configured to control one or more of the at least one spray adjustment actuator to control the droplet size of the pollen or a liquid chemical sprayed by an equivalent number of spray units based at least in part on a magnitude of an air direction angle of the determined air movement direction relative to the ground.

30. The spray vehicle of claim 29, wherein the air direction angle is the angle between the determined air movement direction relative to the ground and a projection of a fore-aft axis of the vehicle onto the ground.

31. The spray vehicle of claim 30, wherein the droplet size is determined at least in part on the air movement speed relative to the ground multiplied by cosine of the air direction angle.
